(19) United States
(12) Patent Application Publication
(10) Pub. No.: US 2014/0088331 A1
(43) Pub. Date: Mar. 27, 2014

(54) PROCESS FOR PRODUCING 1,3-BUTADIENE BY DIMERIZING ETHYLENE AND DEHYDROGENATING THE BUTENES OBTAINED

(71) Applicant: AXENS, Rueil Malmaison Cedex (FR)

(72) Inventor: Gildas ROLLAND, Rueil Malmaison (FR)

(21) Appl. No.: 14/031,063

(22) Filed: Sep. 19, 2013

(30) Foreign Application Priority Data
Sep. 21, 2012 (FR) 12/02509

(51) Int. Cl. C07C 5/48; C07C 2/34

(52) U.S. Cl. CPC C07C 5/48; C07C 2/34
USPC 585/326

(57) ABSTRACT

The present invention describes a process for the production of 1,3-butadiene from ethylene by dimerizing ethylene into butenes using homogeneous catalysis and dehydrogenating the butenes obtained.

The present invention describes a process for the production of 1,3-butadiene from ethylene by dimerizing ethylene into butenes using homogeneous catalysis, then dehydrogenating the butenes obtained.

### PRIOR ART REGARDING BUTADIENE PRODUCTION

Currently, most 1,3-butadiene is produced by extraction of the C4 cut resulting from steam cracking naphtha, generally by extractive distillation using acetonitrile or N-methylpyrrolidone, or by liquid-liquid extraction.

It is also prepared by catalytic dehydrogenation of butenes and of butane (Houdry process, now the CATADIENE™ process from Lummus).

In addition to the dehydrogenation of C4 hydrocarbons into butadiene, another dehydrogenation method (in the presence of oxygen) has gained importance.

Thus, the Phillips O-X-D™ process (oxidative dehydrogenation) for manufacturing butadiene from n-butenes is an example of an industrially exploited dehydrogenation process. The n-butenes, steam and air are reacted at 480-600° C. on a fixed bed catalyst. That process can be used to obtain butene conversions in the range 75% to 80% and to reach a selectivity for butadiene of approximately 88-92%. That process was used by Phillips until 1976.

Petro-Tex has also developed a process for the oxidative dehydrogenation of butenes (Oxo-D™ process). The conversion of oxygen or air is carried out at 550-600° C. on a heterogeneous catalyst (ferrite with Zn, Mn or Mg). By adding steam to control the selectivity, a selectivity for butadiene of 93% (based on the n-butenes) may be obtained with a conversion of 65%.

The most important source of butadiene is still the C4 cut obtained from steam cracking naphtha; the butadiene is produced as a by-product in the production of ethylene. The increase in ethylene production is primarily accommodated by the capacities of steam crackers, for which it has been observed that the feed is changing towards a greater contribution by ethane, because ethane has economic advantages, being available in large quantities and at a lower price compared with other feeds. Because the butadiene production to ethylene ratio decreases when the steam cracker is supplied with feeds containing more ethane, the availability of butadiene is thus increasing less rapidly, resulting in market tension as regards butadiene and the need to diversify supply sources.

Thus, alternative processes for the production of butadiene have been studied. Document JP2011/148720 describes a process for the production of butadiene comprising a step for dimerizing ethylene into butenes on a heterogeneous catalyst followed by oxidative dehydrogenation. The first step, ethylene dimerization, is carried out in the presence of a heterogeneous catalyst constituted by Ni, alumina and silica at a temperature in the range 150° C. to 400° C. The second step, oxidative dehydrogenation of the butenes obtained, is operated between 300° C. and 600° C., in the presence of oxygen and a complex metal oxide comprising molybdenum and bismuth. The innovation of that concatenation of processes resides in the discovery of a more stable heterogeneous dimerization catalyst presenting a low nickel content (less than 1% by weight) and a certain Ni/Al and Si/Al ratio.

That application indicates that the use of the specific catalyst described in JP2011/148720 means that problems with the heterogeneous catalysts which are generally known for that reaction can be minimized, namely reduction of the quantity of isobutene produced during dimerization. In fact, the butene obtained by dimerizing ethylene via a heterogeneous process usually contains quantities of isobutene which are too high for the isobutene to be separated by distillation. The production of isobutene is an unwanted reaction, since isobutene is difficult to separate from the butenes produced. In addition, it is transformed into acrolein during the oxidative dehydrogenation reaction, thus necessitating complex steps for separating and purifying the butadiene. According to JP2011/148720, using a specific catalyst also means that deactivation of the dimerization catalyst by coking can be minimized since, according to JP2011/148720, the step for dimerization by heterogeneous catalysis must be carried out at temperatures above 150° C.

Although the catalyst of JP2011/148720 can be used to minimize the problems encountered with heterogeneous catalysis, it cannot overcome them completely.

The aim of the present invention is to improve the process for the production of butene via ethylene dimerization followed by a dehydrogenation by using a homogeneous catalyst in the first step.

In fact, using a homogeneous catalyst has the advantage of obtaining both a high yield of ethylene over the n-butenes (typically 91% of n-butenes) at the same time as a high conversion per pass (typically 70% to 90%). This minimizes the costs associated with the recycling of unconverted ethylene which characterizes other processes. The process of the invention is thus distinguished from the low conversions per pass and high recycle ratios necessary in heterogeneous processes.

Similarly, the use of a homogeneous catalyst in the dimerization step means that low temperatures can be used (typically below 100° C.). Apart from the obvious advantage of reducing the supply of heat (and thus the costs), this has the following advantages:
operating at low temperatures and with a high conversion per pass (typically 70% to 90%), meaning that a recycle compressor can be dispensed with because the excess ethylene leaves dissolved in the product and a pump can raise the pressure in order to recover the unconverted ethylene, without the need for a chilling cycle because of the small proportion of ethylene to be recovered;
the product from the first step is obtained in the liquid form which can be stored directly: this means that the operability and flexibility of the process as a whole can be enhanced as, if there are problems with the second step, the product from the first step can readily be stored;
dimerization by homogeneous catalysis does not need a means for eliminating coke by burning, as a heterogeneous catalytic bed is not used;
when the variation of the process of the invention is such that the product principally obtained is 1-butene, a portion of the product from the first step can itself be upgraded as is for applications other than those described here (for example as a raw material for copolymerizations of the LLDPE (linear low density polyethylene) or HDPE (high density polyethylene) type because of its very high 1-butene content.

In addition, one of the most prominent advantages of a first step by homogeneous catalysis is the selectivity of the process. The formation of isobutene or other branched compounds which are unfavourable to the second step is practically non-existent in the homogeneous dimerization step. The process is extremely selective towards n-butenes. The term “n-butenes” means 1-butene, cis-2-butene and trans-2-butene.

In particular, isobutene does not have to be eliminated because the n-butenes obtained from dimerization do not contain any.

### Description of the Process

The present invention describes a process for the production of 1,3-butadiene from a stream comprising ethylene, employing the following steps:

a) carrying out a dimerization of the ethylene into n-butenes by bringing said stream into contact with a catalytic system based on a homogeneous catalyst, in order to produce an effluent comprising n-butenes;

b) carrying out a dehydrogenation of the n-butenes obtained in step a) by bringing at least a portion of said effluent into contact with a heterogeneous catalyst in order to produce an effluent comprising 1,3-butadiene.

a/Dimerization of Ethylene into Butenes

The first step of the process of the invention comprises the dimerization of ethylene into butenes in the presence of a homogeneous catalyst.

The dimerization of ethylene into butenes may be carried out using any homogeneous catalytic process known in the art; of these, those which result in a high selectivity for dimers (1-butene or 2-butene) are preferably selected.

In a first variation, ethylene dimerization is carried out in the presence of a catalytic system comprising a catalyst based on alkyl titanate and an aluminium compound with formula AlR₃ or AlR₂H in which R is a hydrocarbyl radical. Catalysts of this type are described in patents FR 2 748 020, FR 2 540 488, FR 2 552 079, FR 2 552 080 and FR 2 748 019. The AlphaButol™ process, marketed by Axens (and described in the review Hydrocarbon Processing, 1984 pp 118-120), is based on this technology and results in the industrial production of 1-butene using a simple layout.

The alkyl titanates used have general formula Ti(OR′)₄ in which R′ is a linear or branched alkyl radical preferably containing 2 to 8 carbon atoms, for example tetraethyl titanate, tetraisopropyl titanate, tetra-n-butyl titanate or tetra-2-ethylhexyl titanate.

Said aluminium compounds used to prepare the catalyst are represented by the general formula AlR₃ or AlR₂H in which each R is a hydrocarbyl radical, preferably alkyl, containing 2 to 6 carbon atoms. The compound AlR₃ is preferred. Examples which may be cited are triethylaluminium, tripropylaluminium, tri-iso-butylaluminium, di-iso-butyl-aluminium hydride and trihexylaluminium.

The components of the catalyst may be brought into contact in a solution of hydrocarbons and/or in 1-butene, produced by dimerization, and/or in one or the by-product(s) of the reaction such as hexenes, preferably in the presence of ethylene. The molar ratio between the aluminium compound and that of the titanate is generally 1:1 to 20:1, preferably in the range 2:1 to 5:1. The concentration of titanium in the prepared solution is advantageously in the range 10⁻⁴ to 0.5 mole per litre, preferably in the range 2×10⁻³ to 0.1 mole per litre.

The temperature at which the catalytic system is prepared is usually in the range −10° C. to +80° C., preferably in the range −10° C. to +45° C. When the ethylene is present in the medium, its quantity preferably corresponds to saturation of the solution at the temperature under consideration and at the selected pressure, 0.1 MPa or more. The catalyst solution obtained may be used as is, or it may be diluted by adding reaction products.

Advantageously, the catalytic system results from the interaction of a preformed mixture of at least one alkyl titanate and at least one ether, with at least one aluminium compound as defined hereinabove. It has been observed that adding ether can reduce the quantity of polymer formed during conversion.

Monoethers or polyethers may be employed as the ethers. Examples which may be used are diethylether, diisopropylether, dibutylether, methyl-t-butylether, tetrahydrofuran, 1,4-dioxane, dihydropyran, and ethylene glycol dimethyl ether. Preferred ethers are tetrahydrofuran and/or 1,4-dioxane. They are used alone or as a mixture.

Particularly preferably, the ether and the alkyl titanate are used in a molar ratio of 1:0.5 to 10:1. Without wishing to be bound by a particular theory, the ether may be considered to form a complex with the titanium, which means that it can be hexacoordinated. If the ether is employed in ratios of more than 10 moles of ether per mole of titanium, for example 20 and higher, or if it is used as a reaction solvent, the reaction is observed to slow down considerably and its selectivity is poorer.

In a first variation, the catalytic system further comprises an additive constituted by at least one quaternary ammonium salt. It has been observed that adding an additive of this type means that the quantity of polymer formed during conversion can be reduced.

The quaternary ammonium salts used have general formula [(R₁R₂R₃R₄)N⁺]X⁻ in which R₁, R₂, R₃ and R₄ are hydrocarbyl radicals, which may be identical or different, for example alkyl, cycloalkyl, aryl, cycloalkyl or aryl radicals substituted with an alkyl group, containing 1 to 30 carbon atoms, and X is a monovalent anion, for example a halide or a hydroxide. Examples which may be cited are tetraethylammonium chloride, tetraethylammonium bromide, trimethyl-cetylammonium chloride, trimethyl-cetylammonium bromide, dimethyl-dilaurylammonium chloride, methyl-trioctylammonium chloride, methyl-tridecylammonium chloride, and benzyl-dimethyl-cetylammonium chloride. The preferred salts are the bromides.

The quantity of quaternary ammonium salts employed during the dimerization reaction may represent 1 part per million by weight (ppm) to 5% by weight, advantageously 1 ppm by weight to 1% by weight, preferably 20 ppm by weight to 5000 ppm by weight, compared with the 1-butene produced, irrespective of whether this quantity is introduced during the reaction (continuous process) or into the vessel before the reaction (batch process).

In a second variation, in addition to a catalyst based on alkyl titanate and an aluminium compound and the optional presence of an ether, the catalytic system additionally comprises an additive constituted by at least one polyethylene glycol, a polyethylene glycol monoether and/or a polyethylene glycol monoester. It has been observed that adding an additive of this type means that the quantity of polymer formed during the conversion can be reduced.

The polyethylene glycols and their monoethers used in accordance with the invention have general formula H(O—CH₂—CH₂)ₙOR″ in which R″ is a hydrogen atom or a hydrocarbyl radical, preferably alkyl, containing 5 to 30 carbon atoms, and n is a whole number from 4 to 30. The polyethylene glycol monoesters have the formula H(OCH₂CH₂)ₙO—(C═O)—R″. The monoethers and monoesters are the preferred derivatives. Examples which may be cited are polyethylene glycols with a molecular mass of 200 to 10000 or more, polyethyleneglycol monolaurylethers, polyethyleneglycol monostearylethers, polyethyleneglycol monolaurates and polyethyleneglycol monostearates. The quantity of polyethylene glycols and/or polyethylene glycol derivatives used during the dimerization reaction may represent 1 part per million by weight (ppm) to 5% by weight, advantageously 1 ppm by weight to 1% by weight, preferably 20 ppm by weight to 5000 ppm by weight, with respect to the 1-butene produced, irrespective of whether this quantity is introduced during the reaction (continuous process) or into the vessel before the reaction (batch process).

The quaternary ammonium salt or polyethylene glycol type additives and/or their derivatives may be used as is or in the form of a solution in a hydrocarbon medium selected from the group formed by hydrocarbons and/or by 1-butene, produced by dimerization, and/or by one or more by-product(s) of the reaction such as hexenes.

Irrespective of whether the process is continuous or is a batch process, the additives of the quaternary ammonium salt or polyethylene glycol type and/or their derivatives, pure or in solution, may be introduced before proceeding to the ethylene dimerization reaction; as an example, they may be used to carry out a passivation treatment of the walls of the reaction vessel prior to starting up the reaction. The walls of the vessel are metallic (metals, steels, alloys, etc.) and may have been subjected to protective treatments (polishing, vitrification, etc.), or they may have under gone anodic protection.

Passivation is carried out using any of the known techniques. Advantageously, the vessel is charged with a solution of 20 ppm to 5% by weight of additive in a hydrocarbon medium, contact being maintained, preferably with stirring, for 10 min to 10 h, preferably 30 min to 3 h, at a temperature below the boiling point of the solvent, generally 20° C. to 100° C., preferably 30° C. to 80° C. The solution is then generally evacuated.

The additives, pure or in solution, may also be introduced in a continuous or batchwise manner as the reaction is under way, for example as a mixture with the titanate solution, preferably in the form of a stream which is independent of the catalyst stream. It may be advantageous to combine a prior passivation treatment of the reaction vessel followed by a continuous or batchwise injection as the reaction is under way.

The ethylene dimerization reaction using a homogeneous catalyst may be carried out at a temperature of 20° C. to 100° C., preferably 30° C. to 60° C. The pressure is generally in the range 1 MPa to 7 MPa, preferably in the range 1.5 MPa to 5 MPa. The contact time is generally 0.3 to 20 h, preferably in the range 0.5 to 10 h.

In a second variation, the ethylene dimerization is carried out in the presence of a catalytic system composed of a nickel compound and an aluminium compound. Catalytic systems of this type have been described in patents U.S. Pat. No. 3,485,881 and U.S. Pat. No. 4,242,531.

In this variation, ethylene dimerization is carried out in the presence of a catalytic system formed by a mixture of a first nickel compound selected from the group comprising the following formulae: (R₃P)₂NiY₂, (R₃PO)₂NiX₂, (R₃AsO)₂NiX₂, (pyridine)₂NiX₂, (bipyridine)NiX₂, (phenanthroline)NiX₂ and a complex formed by a bicyclic nitrogen-containing compound with NiX₂, where R is a hydrocarbon group containing up to 20 carbon atoms, X is a halogen, Y is selected from halogens or hydrocarbon groups containing up to 20 carbon atoms and the pyridine, bipyridine and phenanthroline may or may not be substituted with one or more hydrocarbon groups, and a second compound represented by the formula R′_{z}AlX_{y} where x and y are whole numbers in the range 1 to 3, R′ is a hydrocarbon group containing up to 20 carbon atoms and X is a halogen. The ratio between the first compound (Ni) and the second compound (Al) is generally in the range 1:0.5 to 1:20.

In this second variation, the dimerization reaction is carried out at a temperature in the range −80° C. to 100° C., preferably in the range −20° C. to 50° C. The pressure is generally in the range 0.2 MPa to 14 MPa, and is preferably in the range 0.2 to 3.5 MPa. The contact time for the feed with the catalyst is generally in the range 0.002 h to 20 h, preferably in the range 0.1 h to 2 h. The proportion of catalyst in the reaction mixture is generally in the range 0.001 mole to 0.3 mole of nickel complex per mole of olefin in the feed, preferably in the range 0.005 mole to 0.1 mole of nickel complex per mole of olefin in the feed.

The effluent from the reactor is sent to a system of distillation columns which can be used to separate on the one hand the n-butenes from the ethylene, which is sent to the reactor, and on the other hand the other by-products containing 5 or more carbon atoms, and any solvent such as, for example, a saturated hydrocarbon or a mixture of n-butenes with a composition preferably close to or identical with that of the effluent and a portion of which can be returned to the catalyst preparation section. The foot of the column containing the catalyst, the heavy by-products and the additive can be incinerated, or the recovered catalyst is recycled.

Irrespective of the variation of the process of the invention which is put into practice, the conversion per pass is generally in the range 80% to 90%, and the selectivity for n-butenes is generally in the range 80% to 95%.

In the case of the first variation, the ethylene dimerization is carried out in the presence of a catalytic system comprising a catalyst based on alkyl titanate and an aluminium compound with formula AlR₃ or AlR₂H in which each R is a hydrocarbyl radical. The conversion per pass is then generally 80% to 90%. The selectivity for 1-butene is generally in the range 90% to 94%. 1-butene is obtained in a purity in the range 99.0% to 99.7% by weight. The effluent generally contains less than 0.2% by weight of isobutene, or even less than 0.1% by weight of isobutene. The effluent obtained thereby may thus be sent to the oxidative dehydrogenation step b).

In the case of the second variation, the dimerization is carried out with a catalytic system comprising a catalyst based on nickel and an alkyl aluminium. The conversion per pass is then generally in the range 85% to 90%. The selectivity for n-butenes (1-butene and 2-butene) is generally in the range 80% to 95%. Essentially, cis and trans 2-butene mixed with a small proportion of 1-butene is obtained.

The mixture of n-butenes generally comprises more than 99.5% by weight of n-butenes, of which at least 97% by weight is cis and trans 2-butene, and less than 0.5% by weight, or even less than 0.2% by weight, of isobutene. The mixture obtained thereby may thus be sent to the oxidative dehydrogenation step b).

The effluent from ethylene dimerization thus principally contains n-butenes, as well as small quantities of unreacted ethylene and oligomer, in particular C6 and C8. The effluent contains very small quantities of isobutene.

b/Oxidative Dehydrogenation of n-butenes to 1,3-butadiene

The second step of the process of the invention comprises dehydrogenation of the n-butenes obtained in step a) into 1,3-butadiene in the presence of a heterogeneous catalyst.

Preferably, b) an oxidative dehydrogenation of the n-butenes obtained in step a) is carried out by bringing at least a portion of said effluent into contact with a heterogeneous catalyst in the presence of oxygen and steam in order to produce an effluent comprising 1,3-butadiene.

The oxidative dehydrogenation of n-butene is a reaction between n-butene and oxygen which produces 1,3-butadiene and water.

In addition to the feed of n-butenes, the oxidative dehydrogenation step requires the addition of oxygen. The source of oxygen may be pure oxygen, air or oxygen-enriched air.

The oxidative dehydrogenation reaction is preferably carried out in the presence of steam. Steam can be used to activate the catalyst, remove coke from the catalyst via a water gas reaction, or act as a dissipater for the heat of reaction.

The oxidative dehydrogenation of butenes into butadiene may be carried out using any catalytic process which is known in the art; preferably those which result in a high selectivity for butadiene are selected.

The catalysts used in the oxidative dehydrogenation of n-butene which are currently known are catalysts based on transition metal oxides, in particular catalysts based on oxides of molybdenum and of bismuth, based on ferrite or based on oxides of tin and of phosphorus. The catalysts may or may not be deposited on a support.

In a first variation, the oxidative dehydrogenation is carried out in the presence of a catalytic system comprising a catalyst based on oxides of molybdenum and of bismuth. These catalysts are particularly suitable for oxidative dehydrogenation and are generally based on a Mo—Bi—O multimetallic oxide system which also generally includes iron.

In general, the catalyst also comprises supplemental components from groups 1 to 15 of the periodic classification of the elements, for example potassium, magnesium, zirconium, chromium, nickel, cobalt, cadmium, tin, lead, germanium, lanthanum, manganese, tungsten, phosphorus, cerium, aluminium or silicon.

The composition of a multitude of multimetallic oxide catalysts which are suitable for oxidative dehydrogenation may be included in the general formula:

Mo₁₂BiₐFe_{b}Co_{c}Ni_{d}CrₑX¹_{f}K_{g}Oₓ (I)

in which the variables are each defined as follows: X¹═O, Sn, Mn, La, Ce, Ge, Ti, Zr, Hf, Nb, P, Si, Sb, Al, Cd and/or Mg, a=0.5 to 5, preferably 0.5 to 2, b=0 to 5, preferably 2 to 4, c=0 to 10, preferably 3 to 10, d=0 to 10, e=0 to 10, preferably 0.1 to 4, f=0 to 5, preferably 0.1 to 2, g=0 to 2, preferably 0.01 to 1, and x=a number which is determined by the valency and frequency of the elements of (I) other than oxygen. Examples of multi-component catalysts based on bismuth molybdate which may be used are mixed oxide type catalysts which are compounds of Ni, Cs, Bi, Mo (described in the document M W J. Wolfs, Ph.A. Batist, J. Catal., Vol. 32, p 25 (1974)), mixed oxide type catalysts which are compounds of Co, Fe, Bi, Mg, K, Mo (described in U.S. Pat. No. 3,998,867), mixed oxide catalysts which are compounds of Ni, Co, Fe, Bi, P, K, Mo (described in U.S. Pat. No. 3,764,632), mixed oxide catalysts which are compounds of four metallic elements comprising a divalent metallic cationic component (for example Ca, Mg, Fe, Mn, Sr, Ni), a trivalent metal cation component (for example Fe, Al), Bi and Mo (described in WO2008/147055).

Other suitable catalysts have been described in documents US2008/0119680 or U.S. Pat. No. 4,423,281 such as Mo₁₂BiNi₈Ph_{0.5}Cr₃K_{0.2}Oₓ, Mo₁₂BiNi₇Al₃Cr_{0.5}K_{0.5}Oₓ, Mo₁₂BiNi₆Cd₂Cr₃P_{0.5}Oₓ, Mo₁₂BiNi_{0.5}Cr₃P_{0.5}Mg_{7.5}K_{0.1}Oₓ+SiO₂), Mo₁₂BiCo_{4.5}Ni_{2.5}Cr₃P_{0.5}K_{0.1}Oₓ, Mo₁₂BiFe_{0.1}Ni₈ZrCr₃K_{0.2}Oₓ, Mo₁₂BiFe_{0.1}Ni₈AlCr₃K_{0.2}Oₓ, Mo₁₂BiFe₃Co_{4.5}Ni_{2.5}P_{0.5}K_{0.1}Oₓ+SiO₂), (Mo₁₂BiFe₃Co_{4.5}Ni_{2.5}Cr_{0.5}K_{0.1}Oₓ and Mo_{13.75}BiFe₃Co_{4.5}Ni_{2.5}Ge_{0.5}K_{0.8}Oₓ.

It is also possible to use the catalysts described in JP2011/148720 using a Mo—Bi—O multimetallic oxide system which comprises molybdenum, bismuth, iron and cobalt.

In a second variation, the oxidative dehydrogenation is carried out in the presence of a catalytic system comprising a catalyst based on ferrite. This type of catalyst has been of application in the Oxo-D™ process from Petro-Tex, described in the document Welch L. M., Groce L. J., Christmann H. F., Hydrocarbon Processing, 131, November 1978.

Examples of ferrite-based catalysts are MgFe₂O₄, CoFe₂O₄, CuFe₂O₄, MnFe₂O₄, ZnFe₂O₄, ZnCrFe₂O₄, and MgCrFe₂O₄.

In a third variation, the oxidative dehydrogenation is carried out in the presence of a catalytic system comprising a catalyst based on oxides of tin and of phosphorus. This type of catalyst has been of application in the O-X-D™ process from Phillips Petroleum Company (described in U.S. Pat. No. 3,320,329).

Catalysts based on oxides of tin and of phosphorus (routinely known as Sn—P—O catalysts) may include supplemental components. Examples of catalysts which may be cited are Mg—Sn—P—O, Ba—Sn—P—O (described in U.S. Pat. No. 3,789,078) or Ca—Sn—P—O, Na—Sn—P—O, K—Sn—P—O and Rb—Sn—P—O (described in U.S. Pat. No. 3,925,499) and Li—Sn—P—O (described in the document Hutson T., Skinner R. D., Logan R. S., Hydrocarbon Processing, 133, June 1974).

It is also possible to use combinations of the various types of catalyst as described, such as that described, for example, in EP 2 256 101.

The oxidative dehydrogenation reaction may be carried out at a temperature of 300° C. to 650° C., preferably 310° C. to 550° C., and more preferably 320° C. to 460° C. The pressure is generally 0.01 MPa to 2 MPa, preferably 0.01 MPa to 0.5 MPa, and more preferably 0.05 MPa to 0.3 MPa. The mass flow rate of feed with respect to the mass of the catalyst bed (WHSV) is generally 0.1 to 10 h⁻¹, preferably 0.2 to 5 h⁻¹.

The oxygen (in the O₂ form)/n-butenes molar ratio is generally in the range 0.5 to 0.75, preferably in the range 0.55 to 0.70.

The steam/n-butenes molar ratio is generally 10 to 20:1.

The n-butenes conversion is generally 75% to 85% by weight and the 1,3-butadiene yield is generally 60% to 85%.

The oxidative dehydrogenation may be carried out in any type of reactor such as fixed bed, ebullated bed or moving bed reactors. Preferably, a fixed bed reactor is used.

The oxidative dehydrogenation reaction may be carried out in a reaction section which may comprise one or more reactors, the reactors possibly being identical or different.

c/Separation (Optional Step)

The effluent from oxidative dehydrogenation is a cut which is highly enriched in butadiene. It may contain traces of unreacted n-butenes. The effluent comprising 1,3-butadiene obtained in step b) preferably undergoes at least one separation step in order to obtain a fraction which is enriched in 1,3-butadiene.

Preferably, the effluent comprising 1,3-butadiene can undergo separation by distillation, extractive distillation, solvent extraction or indeed a combination of these techniques. These processes are known to the skilled person. Separation techniques of this type are, for example, described in “Butadiene, Product Stewardship Guidance Manual”, revised Mar. 10, 2002 available at http://www.dow.com/productsafety/pdfs/butadiene_guide.pdf.

Distillation can be envisaged as long as the conversion per pass of 1-butene is sufficient considering the very close boiling points of 1-butene and 1,3-butadiene.

The principal extractive agents used in the extractive distillation are N-methyl-2-pyrrolidone (NMP), dimethylformamide (DMF), acetonitrile or an aqueous methoxy-propionitrile (MOPN)/furfural solution.

Solvent extraction means that the butadiene can be extracted in the solvent constituting the extract. The butenes fraction is insoluble in the solvent. Preferably, solvents such as N-methyl-2-pyrrolidone (NMP), dimethylformamide (DMF) or acetonitrile (ACN) are used.

Thus, butadiene is obtained in a purity of more than 99%.

### BRIEF DESCRIPTION OF DRAWINGS

FIG. 1 illustrates a preferred embodiment of the process of the invention. The facility and the process of the invention will essentially be described.

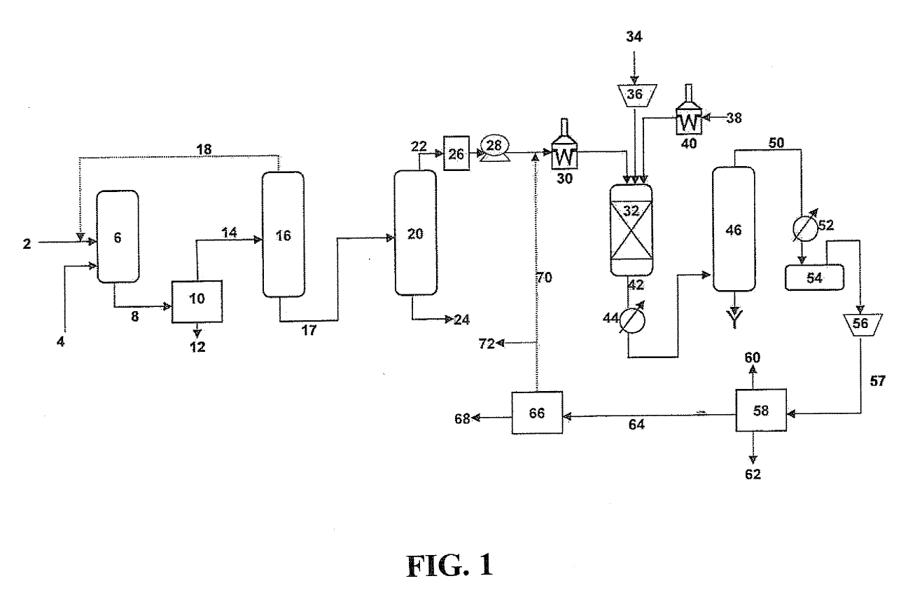

The ethylene and the catalytic system based on a homogeneous catalyst are introduced into the dimerization reactor 6 via the lines 2 and 4 respectively. The reactor 6 may be provided with the usual stirring and cooling systems. The n-butenes production reaction occurs in the liquid phase in the reactor 6 as described in step a). In the reactor 6, the temperature can be maintained between 20° C. and 100° C. and the pressure may be maintained at a value sufficient for all of the compounds to be found in the liquid phase. The effluent evacuated from the reactor 6 via the line 8 contains the catalytic system, the n-butenes, small quantities of unreacted ethylene and oligomers (in particular C6 and C8). The effluent is sent via the line 8 to a catalyst separation system 10, used to separate the spent catalyst and the hydrocarbons contained in the effluent 8, for example by injecting ammoniacal water or an amine. The spent catalyst is evacuated from the system 10 via the line 12. A portion of the catalyst may also be recycled to the dimerization reactor (not shown in FIG. 1). In the system 10, after complete condensation the separated hydrocarbon fraction is pumped via the line 14 to a fractionation section to enrich the hydrocarbon fraction in n-butenes. As an example, the fractionation section is composed of one or more distillation columns that can be used to upgrade and fractionate the hydrocarbon fraction. Referring to FIG. 1, two distillation columns 16 and 20 are used. The hydrocarbon fraction is introduced into the first distillation column 16 via the line 14. The column 16 can be used to obtain a gaseous fraction which is enriched in unconverted ethylene, which can be recycled via the line 18 for introduction into the dimerization reactor 6. The heavy effluent obtained from the bottom of the first distillation column 16 and containing n-butenes and oligomers is sent to the second distillation column 20 via the line 17. A column of the second group of distillation columns 20 can be used to recover a column head fraction enriched in n-butenes via the line 22, for example high purity 1-butene. The oligomers formed in the reactor are evacuated in the liquid fraction from the bottom of the column 20 via the line 24. An optional storage vessel 26 can be used for any storage of the n-butenes 22 obtained at the head of the column 20.

In order to carry out the oxidative dehydrogenation as described in step b), the n-butenes obtained by dimerizing ethylene and optionally stored in the vessel 26 are pressurized by the pump 28, preheated in the furnace 30 and introduced into the oxidative dehydrogenation reactor 32. A stream containing oxygen, for example air, pressurized via the compressor 36 is also introduced via the line 34, as well as steam, preheated in the furnace 40, which is introduced via the line 38. The oxidative dehydrogenation reaction is generally carried out at a temperature of 300° C. to 650° C., at a pressure of 0.01 MPa to 2 MPa, and at a weight hourly space velocity, WHSV, of 0.1 to 10 h⁻¹. The oxidative dehydrogenation may be carried out in any type of reactor such as fixed bed, ebullated bed or moving bed reactors. The reactor shown in the figure is a fixed bed reactor. The oxidative dehydrogenation reaction may be carried out in a reaction section which may comprise one or more reactors, the reactors possibly being identical or different.

The effluent evacuated from the oxidative dehydrogenation reactor via the line 42 contains 1,3-butadiene as well as small quantities of unreacted n-butenes, polymers and light gases (hydrogen and light C2, C3 hydrocarbons). This effluent could be cooled and compressed.

As an example, referring to FIG. 1, the effluent moving in the line 42 is cooled in a system of exchangers 44 then sent to a quenching system 46. The effluent 50 from the quench is cooled in a system of heat exchangers 52, collected in the vessel 54 and compressed in the compressor 56 to obtain a cooled and compressed effluent moving in the line 57.

The effluent 57 containing 1,3-butadiene may optionally undergo a separation step as described in step c). The separation may be carried out by distillation, extractive distillation, solvent extraction or by a combination of these techniques.

As an example, referring to FIG. 1, the effluent is introduced into a separation system 58 via the line 57 to separate the light gases evacuated via the line 60, the polymers formed via the line 62 and a stream which is enriched in 1,3-butadiene in the liquid form evacuated via the line 64.

The stream enriched in 1,3-butadiene containing traces of unreacted n-butenes and obtained via the line 64 may also undergo a separation step in the vessel 66, for example by solvent extraction, in order to recover high purity 1,3-butadiene via the line 68. At least a portion of the n-butenes recovered during the separation step in the vessel 66 may advantageously be recycled to the oxidative dehydrogenation step via the line 70 in order to supply the reactor 32. Alternatively, at least a portion of the n-butenes may be withdrawn for any other use via the line 72.

The following example illustrates the invention without in any way limiting its scope:

### EXAMPLE 1

The feed used in this example mainly comprised ethylene from a steam cracker. It had the following composition:

| | | |
|---|---|---|
| Ethylene | vol % | 99.95 |
| Saturated paraffins (including methane, ethane) | vol % | 0.05 |

The feed underwent dimerization using a homogeneous catalyst. The effluent from the dimerization unit was fractionated in order to separate the gaseous hydrocarbons and the C6+ hydrocarbons from the n-butenes. Water and air were then added to the n-butenes in order to constitute the feed for the oxidative dehydrogenation unit.

In the homogeneous catalysis dimerization unit, the catalytic system comprised a catalyst based on a tetra-n-butyl-tetrahydrofuran titanate complex and an activator co-catalyst which comprised triethylaluminium. The molar ratio between the tetrahydrofuran and the titanate was equal to 2.1:1 and 5 volumes of triethylaluminium were used per volume of tetra-n-butyl titanate. A residence time of 7.0 h was used. The reactor was operated at 55° C. and 2 MPa.

In the dehydrogenation step, the catalytic system comprised a catalyst based on bismuth and molybdenum (Co₉Fe₃Bi₁Mo₁₂O₅₁) prepared using the process described in patent application EP-A-2 256 101, operated at a HSV of 2 h⁻¹, expressed with respect to the liquid feed C4, a pressure of 0.15 MPa and a temperature of 430° C.

| | | | Feed, | Effluent, |
|---|---|---|---|---|
| Mass | | Effluent, | oxidative | oxidative |
| (base 100 for | | ethylene | dehydro- | dehydro- |
| ethylene of | Ethylene | dimerization | genation | genation |
| the feed) | (feed) | unit | unit | unit |
| Ethylene | 100 | See Various | 0 | 0 |
| | | gases | | |
| 1,3-butadiene | 0 | 0 | 0 | 63.6 |
| n-butenes | 0 | 90.9 | 90.9 | Total 469.0 |
| (of which | | | | |
| 1-butene: | | | | |
| 99.5%) | | | | |
| C6+ | 0 | 7.3 | 0 | |
| Various gases | 0 | 1.8 | 0 | |
| Air (1) | 0 | 0 | 138.7 | |
| Steam (2) | 0 | 0 | 303.0 | |
| Other | 0 | 0 | 0 | |

(1)oxygen (in O₂ form)/n-butenes molar ratio = 0.60
(2)steam/n-butene molar ratio = 10

The yield of n-butenes from the first step was thus 90.9% by weight, and the yield from the second step was 70%. The overall butadiene yield from ethylene was 63.6% by weight.

In patent application JP2011/148720, the best 1-butene yield was obtained for the first step of Example 7 (1-butene yield 39.3, conversion=49.5% and selectivity=79.4%). The yield from the second step (oxidative dehydrogenation) was 85.5% (conversion=90% and selectivity=95%), giving an overall maximum yield for the examples of application JP2011/148720 of 33.6%, much lower than the yield for the process of the invention.

### EXAMPLE 2

The feed used in this example mainly comprised ethylene from a steam cracker. It had the following composition:

| | | |
|---|---|---|
| Ethylene | vol % | 99.95 |
| Saturated paraffins (including methane, ethane) | vol % | 0.05 |

The feed underwent dimerization using a homogeneous catalyst. The effluent from the dimerization unit was fractionated in order to separate the gaseous hydrocarbons and the C6+ hydrocarbons from the n-butenes. Water and air were then added to the n-butenes in order to constitute the feed for the oxidative dehydrogenation unit.

In the homogeneous catalysis dimerization unit, the catalytic system comprised a catalyst based on a nickel (II) chloride complex: bis(tributylphosphine)dichloronickel, and a co-catalyst activator which was ethylaluminium dichloride. The molar ratio between the tributylphosphine and the nickel was equal to 1 and the Al:Ni molar ratio was equal to 10. A residence time of 0.5 h was used. The reactor was operated at 50° C. and 1.8 MPa.

In the dehydrogenation step, the catalytic system comprised a catalyst based on bismuth and molybdenum (Co₉Fe₃Bi₁Mo₁₂O₅₁) prepared using the process described in patent application EP-A-2 256 101, operated at a HSV of 2 h⁻¹, expressed with respect to the liquid feed C4, a pressure of 0.15 MPa and a temperature of 430° C.

| | | | Feed, | Effluent, |
|---|---|---|---|---|
| Mass | | Effluent, | oxidative | oxidative |
| (base 100 for | | ethylene | dehydro- | dehydro- |
| ethylene of | Ethylene | dimerization | genation | genation |
| the feed) | (feed) | unit | unit | unit |
| Ethylene | 100 | See Various | 0 | 0 |
| | | gases | | |
| 1,3-butadiene | 0 | 0 | 0 | 54.4 |
| n-butenes | 0 | 83.7 | 83.7 | Total 421.5 |
| (of which | | | | |
| 1-butene: | | | | |
| 99.5%) | | | | |
| C6+ | 0 | 14.5 | 0 | |
| Various gases | 0 | 1.8 | 0 | |
| Air (1) | 0 | 0 | 123.2 | |
| Steam (2) | 0 | 0 | 269.0 | |
| Other | 0 | 0 | 0 | |

(1)oxygen (in O₂ form)/n-butenes molar ratio = 0.60
(2)steam/n-butene molar ratio = 10

The yield of n-butenes from the first step was thus 83.7% by weight, and the yield from the second step was 65%. The overall butadiene yield from ethylene was 54.4% by weight.

In patent application JP2011/148720, the best 1-butene yield was obtained for the first step of Example 7 (1-butene yield 39.3, conversion=49.5% and selectivity=79.4%). The yield from the second step (oxidative dehydrogenation) was 85.5% (conversion=90% and selectivity=95%), giving an overall maximum yield for the examples of application JP2011/148720 of 33.6%, much lower than the yield for the process of the invention.

The entire disclosures of all applications, patents and publications, cited herein and of corresponding French Application No. 12/02509, filed Sep. 21, 2012 are incorporated by reference herein.

1. A process for the production of 1,3-butadiene from a stream comprising ethylene, employing the following steps:
a) carrying out a dimerization of the ethylene into n-butenes by bringing said stream into contact with a catalytic system based on a homogeneous catalyst, in order to produce an effluent comprising n-butenes;
b) carrying out a dehydrogenation of the n-butenes obtained in step a) by bringing at least a portion of said effluent into contact with a heterogeneous catalyst, in order to produce an effluent comprising 1,3-butadiene.

2. A process according to claim 1, in which the ethylene dimerization is carried out in the presence of a catalytic system comprising a catalyst based on alkyl titanate and an aluminium compound with formula AlR₃ or AlR₂H in which each R is a hydrocarbyl radical.

3. A process according to claim 2, in which the catalytic system results from the interaction of a preformed mixture of at least one alkyl titanate and at least one ether with at least one aluminium compound with formula AlR₃ or AlR₂H in which each R is a hydrocarbyl radical.

4. A process according to claim 3, in which the ether and the alkyl titanate are used in a molar ratio of 1:0.5 to 10:1.

5. A process according to claim 2, in which the catalytic system further comprises an additive constituted by at least one quaternary ammonium salt.

6. A process according to claim 2, in which the catalytic system further comprises an additive constituted by at least a polyethylene glycol, a polyethylene glycol monoether and/or a polyethylene glycol monoester.

7. A process according to claim 2, in which the ethylene dimerization is carried out at a temperature of 20° C. to 100° C., at a pressure in the range 1 MPa to 7 MPa and with a contact time in the range 0.3 to 20 h.

8. A process according to claim 1, in which the ethylene dimerization is carried out in the presence of a catalytic system formed by a mixture of a first nickel compound selected from the group comprising the following formulae: (R₃P)₂NiY₂, (R₃PO)₂NiX₂, (R₃AsO)₂NiX₂, (pyridine)₂NiX₂, (bipyridine)NiX₂, (phenanthroline)NiX₂ and a complex formed by a bicyclic nitrogen-containing compound with NiX₂, where R is a hydrocarbon group containing up to 20 carbon atoms, X is a halogen, Y is selected from halogens or hydrocarbon groups containing up to 20 carbon atoms and the pyridine, bipyridine and phenanthroline may or may not be substituted with one or more hydrocarbon groups, and a second compound represented by the formula R′_{z}AlX_{y} where x and y are whole numbers in the range 1 to 3, R′ is a hydrocarbon group containing up to 20 carbon atoms and X is a halogen.

9. A process according to claim 8, in which the ethylene dimerization is carried out at a temperature of −80° C. to 100° C., a pressure in the range 0.2 MPa to 14 MPa and with a contact time in the range 0.002 h to 20 h.

10. A process according to claim 1 in which, in step b), oxidative dehydrogenation of the n-butenes obtained in step a) is carried out by bringing at least a portion of said effluent into contact with a heterogeneous catalyst in the presence of oxygen and steam, so as to produce an effluent comprising 1,3-butadiene.

11. A process according to claim 10, in which the oxidative dehydrogenation is carried out in the presence of a catalytic system comprising a catalyst based on oxides of molybdenum and of bismuth.

12. A process according to claim 10, in which the oxidative dehydrogenation is carried out in the presence of a catalytic system comprising a catalyst based on ferrite.

13. A process according to claim 10, in which the oxidative dehydrogenation is carried out in the presence of a catalytic system comprising a catalyst based on oxides of tin and of phosphorus.

14. A process according to claim 10, in which the oxidative dehydrogenation of the n-butenes is carried out at a temperature of 300° C. to 650° C., at a pressure of 0.01 MPa to 2 MPa and at a weight hourly space velocity of 0.1 to 10 h⁻¹.

15. A process according to claim 1, in which the effluent comprising 1,3-butadiene obtained in step b) undergoes at least one separation step in order to obtain a fraction which is enriched in 1,3-butadiene.
